(19) United States
(12) Patent Application Publication
(10) Pub. No.: US 2025/0191760 A1
(43) Pub. Date: Jun. 12, 2025

(54) MULTIMODAL ANALYSIS COMBINING MONITORING MODALITIES TO ELICIT COGNITIVE STATES AND PERFORM SCREENING FOR MENTAL DISORDERS

(71) Applicant: aiberry, inc., Bellevue, WA (US)

(72) Inventors: Newton Howard, Providence, RI (US); Soujanya Poria, Singapore (SG); Navonil Majumder, Singapore (SG); Sergey Kanareykin, Arlington, MA (US); Sangit Rawlley, Frisco, TX (US); Tanya Juarez, Frederick, MD (US)

(73) Assignee: aiberry, inc., Bellevue, WA (US)

(21) Appl. No.: 18/887,228

(22) Filed: Sep. 17, 2024

Related U.S. Application Data
(63) Continuation of application No. 17/229,147, filed on Apr. 13, 2021, now abandoned.

(51) Int. Cl. G16H 50/20; G06N 20/00; G16H 10/60; G16H 20/70; G16H 40/20; G16H 40/67; G16H 80/00

(52) U.S. Cl. CPC G16H 50/20; G06N 20/00; G16H 10/60; G16H 40/20; G16H 40/67; G16H 80/00; G16H 20/70

(57) ABSTRACT

Embodiments may provide improved techniques for mental health screening and its provision. For example, a method may comprise receiving input data relating to communications among persons, the input data comprising a plurality of modalities, extracting features relating to the plurality of modalities from the received input data, performing multimodal fusion on the extracted features, wherein the multimodal fusion is performed on at least some of the features relating to individual modalities and on at least some combinations of features relating to a plurality of modalities, classifying the fused features using a trained model for detection of at least one mental disorder, and generating a representation of a disorder state based on the classified fused features. For the multimodal fusion, a late fusion scheme instead of early fusion may be used to make the model more interpretable and explainable without compromising the performance.

### CROSS-REFERENCE TO RELATED APPLICATIONS

This application is a continuation of U.S. patent application Ser. No. 17/229,147 filed Apr. 13, 2021, which application claims the benefit of U.S. Provisional Application No. 63/009,082, filed Apr. 13, 2020, the contents of which are incorporated herein in their entirety.

### BACKGROUND

The present invention relates to devices, methods and systems that enable advanced non-invasive screening for mental disorders.

Automated multimodal analysis is gaining increasing interest in the field of mental disorder screening, because it allows optimizing the use of therapist time, and increases the options for monitoring of disorders such as depression, anxiety, suicidal ideation, and post-traumatic stress disorder.

For example, the United States faces a mental health epidemic. Nearly one in five American adults suffers from a form of mental illness. Suicide rates are at an all-time high, and statistics show that nearly 115 people die daily from opioid abuse. Studies have shown that depression makes up around one half of co-occurring disorders. For instance, co-occurring disorders of depression and anxiety are by far the most common psychological conditions in the community, with an estimated 20.9% of US citizens experiencing a major depressive episode and 33.7% suffering from an anxiety disorder at some point throughout their lives. Additionally, there is an extremely high comorbidity between anxiety and depression, with 85% of people diagnosed with depression problems also suffering significant anxiety and 90% of people diagnosed with anxiety disorders suffering significant depression.

Globally, more than 300 million people of all ages suffer from depression, with an astounding 20% increase in a decade. Currently, one in eight Americans over 12 years old take an antidepressant medication every day. Unfortunately, depression can lead to suicide in many instances. Close to 800,000 people die by suicide every year globally and it is the second leading cause of death in 15-29-year-olds.

Although there are known, effective treatments for depression, fewer than half of those affected in the world (in many countries, fewer than 10%) receive such treatments. The economic burden of depression alone is estimated to be at least $210 billion annually, with more than half of that cost coming from increased absenteeism and reduced productivity in the workplace. The nation is confronting a critical shortfall in psychiatrists and other mental health specialists that is exacerbating the crisis. Nearly 40% of Americans live in areas designated by the federal government as having a shortage of mental health professionals; more than 60% of U.S. counties are without a single psychiatrist within their borders. Additionally those fortunate enough to live in areas with sufficient access to mental health services often can't afford them because many therapists don't accept insurance.

The increase in the mental disorders worldwide is an epidemic and the health systems have not yet adequately responded to this burden. As a consequence, a need arises for automated mental health screening and its provision all over the world.

### SUMMARY

Embodiments may provide improved techniques for mental health screening and its provision. For example, an embodiment may include a multimodal analysis system, utilizing artificial intelligence and/or machine learning, in which video footage of the subject is separated into multiple data streams—video, audio, and speech content—and analyzed separately and in combination, to extract patterns specific to a particular disorder. The analysis results may be fused to provide a combined result and one or more scores showing the likelihood that the subject has a particular mental disorder may be assigned. This is an example of a late fusion scheme that may be used to make the model more interpretable and explainable without compromising the performance. Embodiments may include additional modalities that can be integrated as required, to enhance the system sensitivity and improve results.

For example, in an embodiment, a method may be implemented in a computer system comprising a processor, memory accessible by the processor, and computer program instructions stored in the memory and executable by the processor, the method may comprise receiving input data relating to communications among persons, the input data comprising a plurality of modalities, extracting features relating to the plurality of modalities from the received input data, performing multimodal fusion on the extracted features, wherein the multimodal fusion is performed on at least some of the features relating to individual modalities and on at least some combinations of features relating to a plurality of modalities, classifying the fused features using a trained model for detection of at least one mental disorder, and generating a representation of a disorder state based on the classified fused features. For the multimodal fusion, a late fusion scheme instead of early fusion may be used to make the model more interpretable and explainable without compromising the performance.

In embodiments, the plurality of modalities comprises text information, audio information, and video information. The multimodal fusion may be performed on at least some of the text information, audio information, video information, text-audio information, text-video information, audio-video information, and text-audio-video information. The mental disorder may be one of depression, anxiety, suicidal ideation, and post-traumatic stress disorder. The mental disorder may be depression and the representation of the disorder state is a predicted PHQ-9 score or a similar industry-standard metric such as CES-D Depression Scale. The persons may be of any of at least one of age, gender, race, nationality, ethnicity, culture, and language. The method may be implemented as a stand-alone application, integrated with a telemedicine/telehealth platform, integrated with other software, or integrated with other applications/marketplaces that provide access to counselors and therapy. The method may be used for at least one of screening in clinical settings (ER visits, primary care, pre and post-surgery), validating clinical observations (provision of 2nd opinions, expediting complicated diagnostic paths, verifying clinical determinations), screening in the field (at home, school, workplace, in the field), virtual follow up via telehealth scenarios (synchronous—video call with patient, asynchronous—video messages), self-screening for consumer use (triage channels, self-administered assessments, referral mechanisms), screening through helplines (suicide prevention, employee assistance).

In an embodiment, a system may comprise a processor, memory accessible by the processor, and computer program instructions stored in the memory and executable by the processor to perform receiving input data relating to communications among persons, the input data comprising a plurality of modalities, extracting features relating to the plurality of modalities from the received input data, performing multimodal fusion on the extracted features, wherein the multimodal fusion is performed on at least some of the features relating to individual modalities and on at least some combinations of features relating to a plurality of modalities, classifying the fused features using a trained model for detection of at least one mental disorder, and generating a representation of a disorder state based on the classified fused features. The model may discriminate between two speakers in the conversation (e.g., between therapist and patient) and weigh them differently.

In an embodiment, a computer program product may comprise a non-transitory computer readable storage having program instructions embodied therewith, the program instructions executable by a computer, to cause the computer to perform a method that may comprise receiving input data relating to communications among persons, the input data comprising a plurality of modalities, extracting features relating to the plurality of modalities from the received input data, performing multimodal fusion on the extracted features, wherein the multimodal fusion is performed on at least some of the features relating to individual modalities and on at least some combinations of features relating to a plurality of modalities, classifying the fused features using a trained model for detection of at least one mental disorder, and generating a representation of a disorder state based on the classified fused features.

### BRIEF DESCRIPTION OF THE DRAWINGS

The details of the present invention, both as to its structure and operation, can best be understood by referring to the accompanying drawings, in which like reference numbers and designations refer to like elements.

FIG. 1 shows a high level overview of the infrastructure setup along with the services used from the cloud provider (AWS in this case).

FIG. 2 shows the high level view of the system used to separate the modalities from video recording, extract features, and evaluate the data and assign the score values to individual modalities, to produce a combined score.

FIGS. 3a and 3b show the processing pipeline from the infrastructure point of view, as concurrently running on multiple network nodes. Each square represents a microservice that runs independently and performs a highly specialized task. (FIG. 3a shows from Start to step 316, then FIG. 3b continues the pipeline after step 316 to end.)

FIG. 4 shows how in an example embodiment of the system, it displaying the original media, the individual score for each of the analysis modalities, and the final results of the assessment.

FIG. 5 is an exemplary block diagram of a computer system, in which processes involved in the embodiments described herein may be implemented.

FIG. 6 is an exemplary block diagram of how the states of the conversation may be tracked using DialogueRNN workflow as the utterances are being fed, representing global state, speaker state indicating a profile of each individual speaker, and a disorder state.

FIG. 7 is an exemplary block diagram of the DialogueGCN workflow where a dialogue is represented as a graph, followed by a graph convolutional layer to get convoluted features which are used to obtain depression score.

### DETAILED DESCRIPTION

Embodiments may provide improved techniques for mental health treatment and its provision. For example, an embodiment may include a multimodal analysis system, utilizing artificial intelligence and/or machine learning, in which video footage of the subject is separated into multiple data streams—video, audio, and speech content—and analyzed separately and in combination, to extract patterns specific to a particular disorder, and assign one or more scores showing the likelihood that the subject has a particular mental disorder. Embodiments may include additional modalities that can be integrated as required, to enhance the system sensitivity and improve results.

Telepsychiatry is a branch of telemedicine defined by the electronic delivery of psychiatric services to patients. This typically includes providing psychiatric assessments, therapeutic services, and medication management via telecommunication technology, most commonly videoconferencing. By leveraging the power of technology, telepsychiatry makes behavioral healthcare more accessible to patients, rather than patients having to overcome barriers, like time and cost of travel, to access the care they need. Embodiments used as part of the telehealth engagement can clearly be an asset for the provider. Telepsychiatry or telehealth can even expand its scope into the forensic telepsychiatry is the use of a remote psychiatrist or nurse practitioner for psychiatry in a prison or correctional facility, including psychiatric assessment, medication consultation, suicide watch, pre-parole evaluations and more.

Embodiments may be implemented as a standalone application or may be integrated with telemedicine/telehealth platforms utilizing ZOOM®, TELEDOC®, etc. Embodiments may be integrated with other software such as EMR and other applications/marketplaces that provide access to counselors, therapy, etc.

Embodiments may be applied to different use-cases. Examples may include screening in clinical settings (ER visits, primary care, pre and post-surgery), validating clinical observations (provision of 2nd opinions, expediting complicated diagnostic paths, verifying clinical determinations), screening in the field (at home, school, workplace, in the field), virtual follow up via telehealth scenarios (synchronous—video call with patient, asynchronous—video messages), self-screening for consumer use (triage channels, self-administered assessments, referral mechanisms), screening through helplines (suicide prevention, employee assistance). etc.

Embodiments may provide an entire end to end system that uses multimodal analysis for mental disorder screening and analysis. Embodiments may be used for one mental disorder, or for a wide range of disorders. Embodiments may utilize artificial intelligence and/or machine learning models that are specifically trained for identifying markers of mental disorders. Embodiments may utilize analysis modes such as text inference, audio inference, video inference, text-audio inference, text-video inference, audio-video inference, and text-audio-video inference. The multimodal approach may be expanded to address comorbid disorders. Embodiments may be used for multiple use cases outside mental disorders: lie detection in prison environments, malingering in the military/VA environment.

Embodiments may be used across a wide variety of embodiments may be used across all demographics, such as age (children, adults), gender, race, nationality, ethnicity, culture, language, etc., and may include scalable models that can be expanded. Embodiments may be used for initial detection and follow-on analysis (primarily for screening, not final diagnosis). Embodiments may be integrated into existing telehealth systems to increase the accuracy of the analysis and tracking of outcomes. Embodiments may be used to analyze the triggers or changes in behaviors for mental issues (aggregate population data, for example, for a particular hospital system's patients). Embodiments may be used to monitor communications between two parties—both done in person or remotely (telehealth, i.e., therapist/patient). Embodiments may be trained to evaluate monologues as a well as group conversations.

Embodiments may be implemented as an event-based cloud-native system that can be used on multiple devices and not constrained to specific locations (mini-clouds running on individual devices, for on-premises installations, etc.). Embodiments may provide flexibility to use 3rd party applications and APIs and may evolve to keep in line with industry (plug and play). Such APIs may be integrated in other healthcare systems such as EMR. Embodiments may be used as a standalone screening tool and may be required for security reasons (HIPAA).

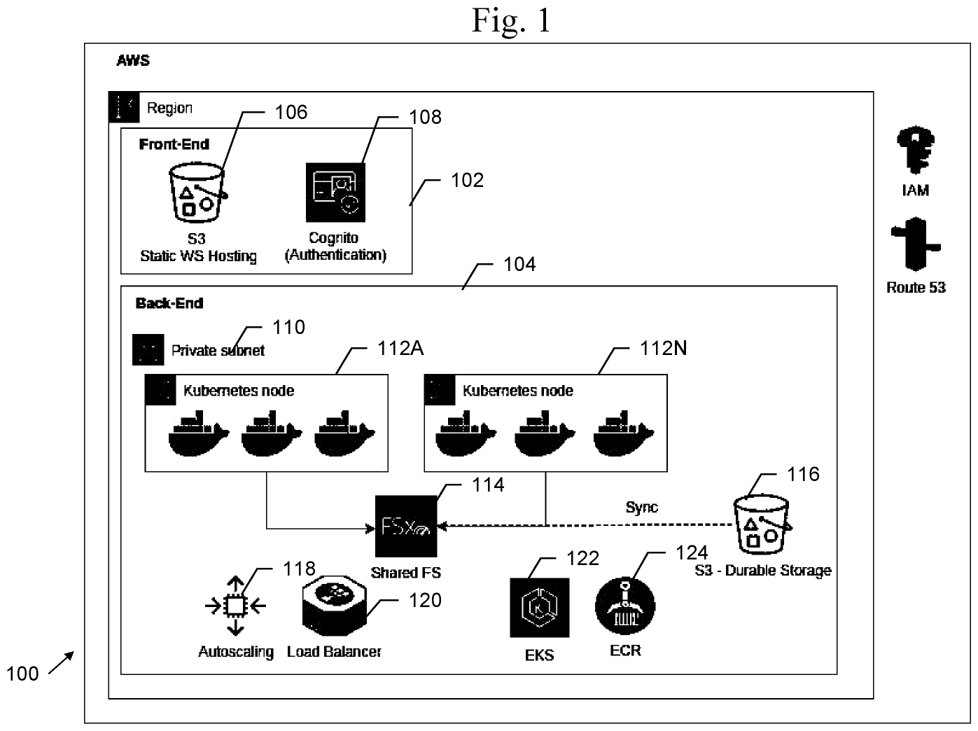

An exemplary block diagram of an embodiment of a system architecture 100 in which the present techniques may be implemented is shown in FIG. 1. System architecture 100 may be implemented, for example, using a cloud service, such as AMAZON WEB SERVICES® (AWS). System architecture 100 may include front end processing 102 and back end processing 104. Front end processing 102 may, for example, be implemented using static website hosting 106 and authentication services 108. Front end processing 102 may include, for example, data input and preprocessing functions. Back end processing 104 may be implemented using a private subnet 110 to provide communications among application processing nodes 112A-N. Application processing nodes 112A-N may share file services 114, as well as other services, such as durable storage 116, autoscaling 118, load balancer 120, Elastic Kubernetes Service 122, and Elastic Container Service 124.

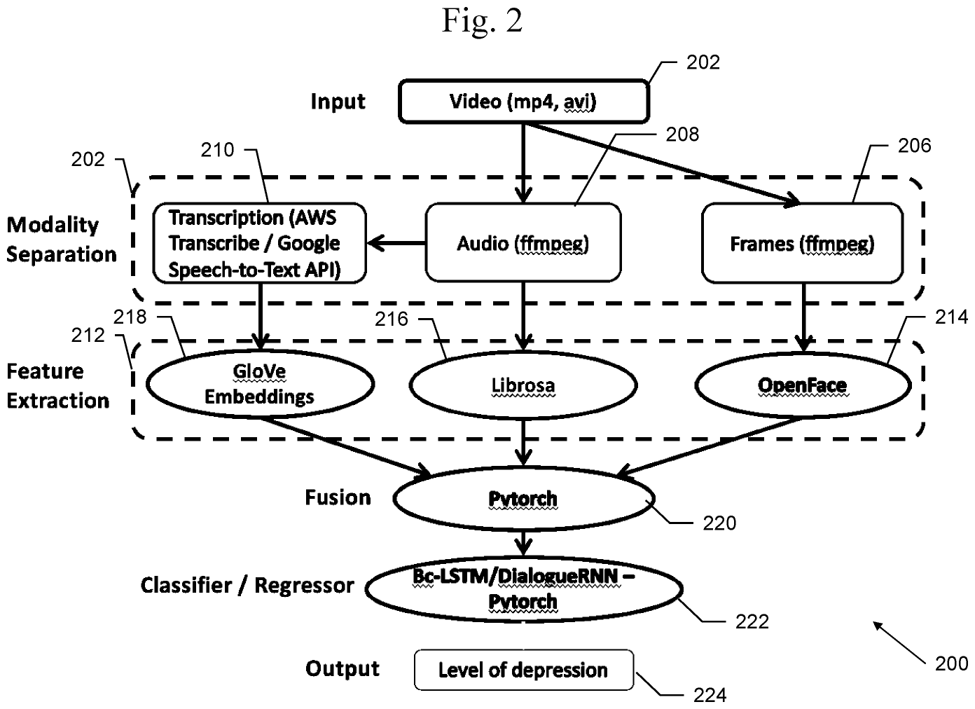

An exemplary embodiment of a process 200 of determining a mental disorder is shown in FIG. 2. In this example, the mental disorder to be determined is depression, but embodiments are applicable to other mental disorders, such as depression, anxiety, suicidal ideation, etc., as well. In addition, embodiments may be used across all demographics, such as age (children, adults), gender, race, nationality, ethnicity, culture, language, etc. Process 200 begins with 202, in which an input stream relating to communications among persons may be obtained. Such an input stream may include channels/modalities such as textual (T), visual (V), and acoustic (A). For example, the input stream may be obtained from sources such as text message/email conversations, video and/or audio recordings of conversations, multi-media presentations or conversations, etc. For example, typical formats of video streams may include mp4, avi, mpeg, etc.

At 204, features from each channel/modality may be separated. For example, frames may be extracted 206 from video streams and audio may be extracted 208 from audio visual streams. Such extraction may be performed by software such as ffmpeg. Extracted audio may be transcribed 210, using a transcription service, such as AMAZON WEB SERVICES® (AWS®) or GOOGLE® Speech-to-Text API.

At 212, features from each channel/modality may be extracted independently. For example, Visual Features may be extracted 214 that constitute facial contour coordinates of the subjects visible in the videos. Software such as the OpenFace toolkit or similar functionality may be used. Acoustic Features may be extracted 216 that constitute MFCC (Mel frequency cepstral coefficients) and mel-spectrogram features of the audio signal. Software such as the Librosa package or similar functionality may be used. Textual Features from text data or from transcribed audio may be extracted 218 using a pretrained model that is fine-tuned for the given mental-disorder detection task to obtain task-specific word-level and utterance-level features. Software such as a pre-trained BERT model or similar functionality may be used.

At 220, multimodal fusion of the extracted features may be performed. Early fusion or data-level fusion involves fusing multiple data before conducting an analysis. Late fusion or decision level fusion uses data sources independently followed by fusion at a decision-making stage. The specific examples shown herein are merely examples, embodiments may utilize either type of fusion. For the multimodal fusion, a late fusion scheme instead of early fusion may be used to make the model more interpretable and explainable without compromising the performance.

Multimodal fusion techniques are employed to aggregate information from the features extracted from channels/modalities such as textual (T), visual (V), and acoustic (A). Embodiments may utilize hierarchical fusion to obtain conversation-level multimodal representation. This approach first fuses two modalities at a time, specifically [T, V], [V, A], and [T, A], and then fuses these three bimodal representations into a trimodal representation [T, V, A]. This hierarchical structure enables the network to compare multiple modalities and resolve conflict among them, yielding densely-informative multimodal representation relevant to the given task. Software such as Pytorch or similar functionality may be used.

At 222, speaker-specific detection of the mental disorder may be performed. Speaker identification may be performed using a trained classifier that looks into a fixed number of initial turns in the input video and identifies the patient. The mental-disorder classifier then evaluates the identified patient based on the full video. Although the detection may be speaker-specific, the classifier or other model used may be non-speaker-specific. Conversation Processing may be performed, utilizing artificial intelligence and/or machine learning, such as neural network processing, which may include, for example, recurrent neural networks (for example, DialogueRNN) and graph convolutional networks (for example, DialogueGCN) to obtain a task-specific representation (disorder state) of each utterance. The input conversation may be fed to the Conversation Processing modules one utterance at a time, along with the associated speaker identification information, in a temporal sequence.

For example, in recurrent neural networks, such as DialogueRNN, three key states for the conversation may be tracked as the utterances are being fed: a global state that represents general context at some time in the conversation, a speaker state indicating a profile of each individual speaker, based on their past utterances, as the conversation progresses, and a disorder state that indicates a given disorder representation of each utterance and that may be calculated based on the corresponding speaker state and global state, along with preceding depression state. Examples of processing, such as may be performed by DialogueRNN are described further below.

In graph convolutional networks, such as DialogueGCN, a conversation may be represented as a graph where each node of the graph corresponds to an utterance. Examples of processing, such as may be performed by DialogueGCN are described further below.

Further, at 222, the disorder representations/states corresponding to the patient may be aggregated into a single/unified representation. This may be fed to a feed-forward network for final disorder score calculation 224, such as a predicted Patient Health Questionnaire (PHQ-9) score or a similar industry-standard metric such as CES-D Depression Scale, which may indicate a level of depression, or other metrics that may indicate levels of other disorders.

Embodiments may utilize a stochastic gradient descent-based Adam optimizer to train the network by minimizing the squared difference between the target depression score and predicted depression score by the network.

Embodiments may utilize a configurable runtime infrastructure including a microservices based architecture and may be designed to execute in cloud native environments benefiting from the cloud provider's security features and optimal use of infrastructure. The provisioning of the infrastructure and the respective microservices may be automated, parameterized and integrated into modern Infrastructure-as-a-Service (IaaS) and Continuous Integration/Code Deployment (CI/CD) pipelines that allow for fast and convenient creation of new and isolated instances of the runtime. As with all cloud native solutions, the security aspects may be governed by the shared responsibility model with the selected cloud vendor. The solution may be built on the principle of least privilege, securing the data while in transit and at rest. Access to data may be allowed only to authorized users and is governed by cloud security policies.

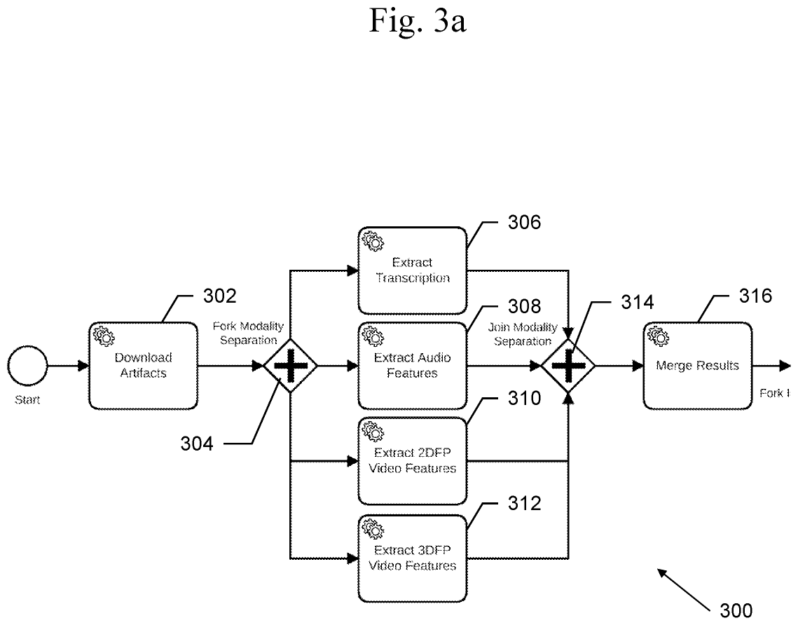

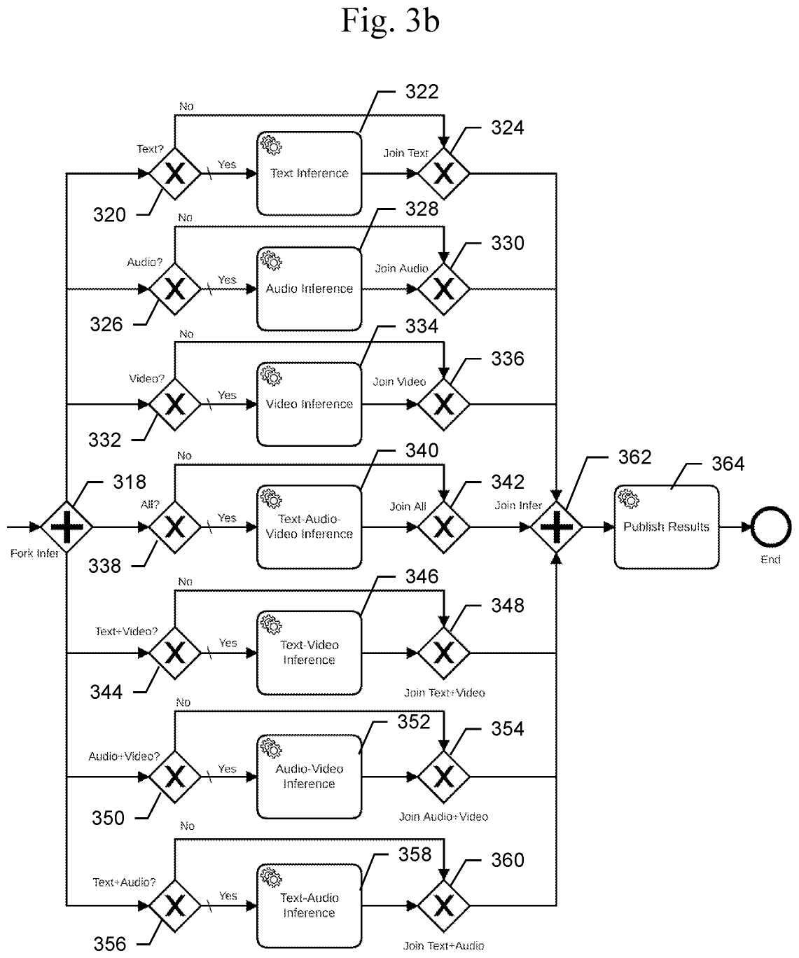

An exemplary embodiment of a process 300 of determining a mental disorder is shown in FIGS. 3a, 3b. Process 300 begins with 302 in FIG. 3a, in which an input stream or artifacts may be obtained or downloaded. Such an input stream may include channels/modalities such as textual (T), visual (V), and acoustic (A). For example, the input stream may be obtained from sources such as text message/email conversations, video and/or audio recordings of conversations, multi-media presentations or conversations, etc. At 304, features from each channel/modality may be separated and extracted. For example, audio transcription 306, audio features 308, two-dimensional video features 310, and three-dimensional video features 312 may be extracted from the separated modalities. At 314, the features extracted from the separated modalities may be joined and at 316, the results merged.

Turning now to FIG. 3b, the merged results 316 may be forked 318 to a plurality of inference processing blocks. For example, at 320, it may be determined whether text is present, and if so, at 322, results relating to, for example, mental disorders may be inferred. Then, at 324, the text inference results may be joined to the text. Likewise, at 326, it may be determined whether audio information, such as voice, is present, and if so, at 328, results relating to, for example, mental disorders may be inferred. Then, at 330, the audio inference results may be joined to the audio information. At 332, it may be determined whether video information is present, and if so, at 334, results relating to, for example, mental disorders may be inferred. Then, at 336, the video inference results may be joined to the video information. At 332, it may be determined whether video information is present, and if so, at 334, results relating to, for example, mental disorders may be inferred. Then, at 336, the video inference results may be joined to the video information. At 338, it may be determined whether text-audio-video information is present, and if so, at 340, results relating to, for example, mental disorders may be inferred. Then, at 342, the text-audio-video inference results may be joined to the text-audio-video information. At 344, it may be determined whether text-video information is present, and if so, at 342, results relating to, for example, mental disorders may be inferred. Then, at 344, the text-video inference results may be joined to the text-video information. At 350, it may be determined whether audio-video information is present, and if so, at 352, results relating to, for example, mental disorders may be inferred. Then, at 354, the audio-video inference results may be joined to the audio-video information. At 356, it may be determined whether text-audio information is present, and if so, at 358, results relating to, for example, mental disorders may be inferred. Then, at 360, the text-audio inference results may be joined to the text-audio information.

At 362, the joined information 324, 330, 336, 342, 348, 354, and 360 may all be joined 362 together to form published results 364.

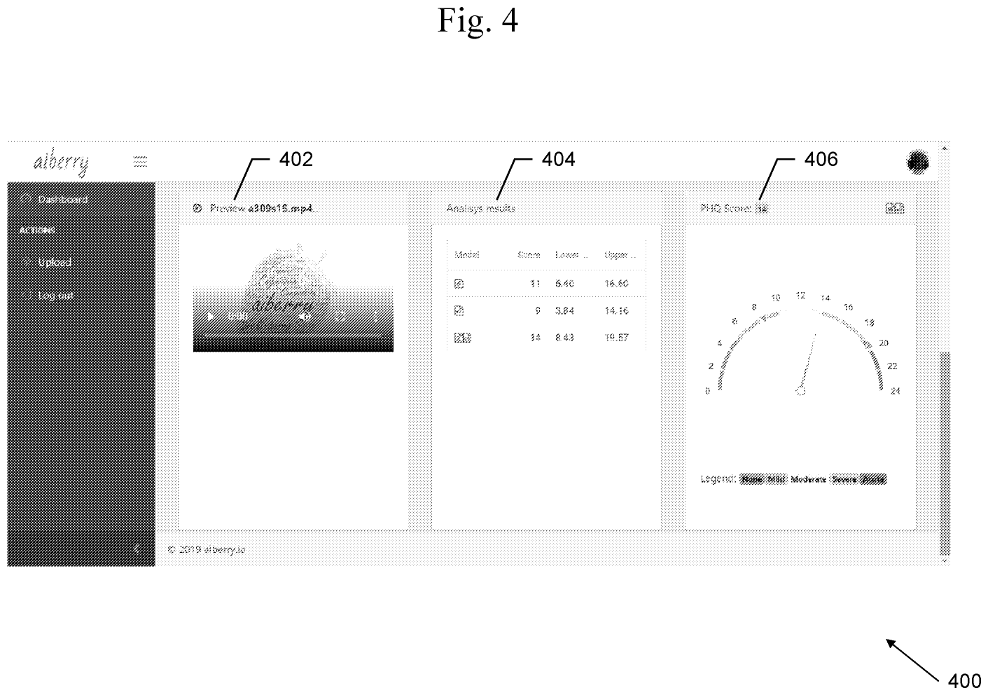

An exemplary screenshot of a user interface 400 in which the present techniques may be implemented is shown in FIG. 4. In this example, user interface 400 may include a preview 402 of the video, audio, text, etc., that is to be analyzed, analysis results 404, and a score 406, such as a disorder score, which may indicate, for example, a level of depression or other mental health condition.

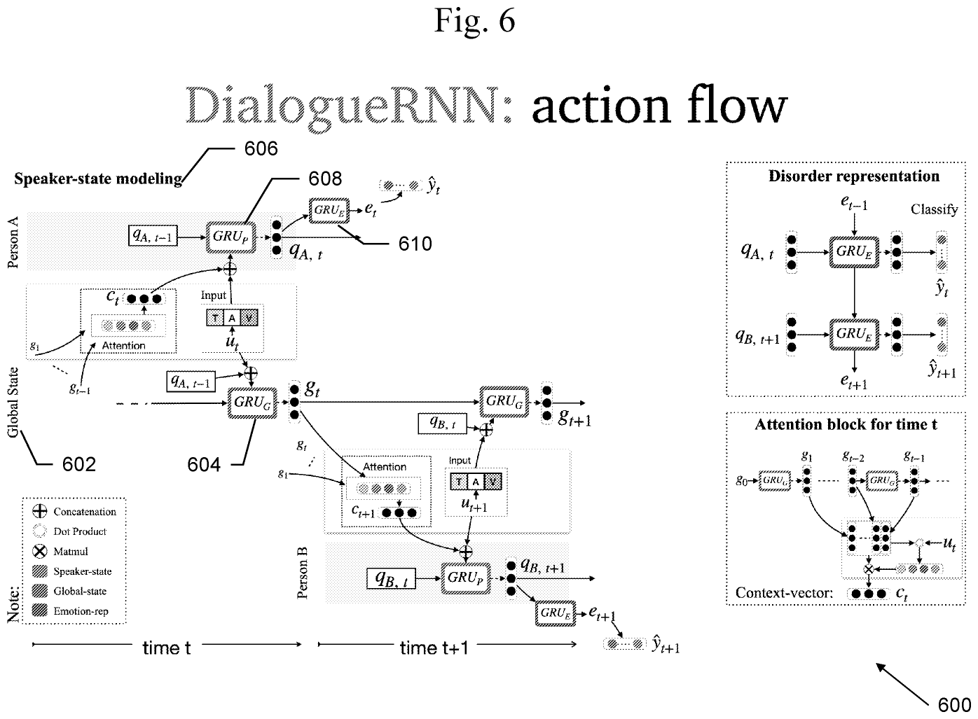

An example of how the states of a conversation may be tracked is shown in FIG. 6. This example uses a DialogueRNN process 600 as the utterances are being fed, representing global state, speaker state indicating a profile of each individual speaker, and a disorder state.

Global state (Global GRU) 602 aims to capture the context of a given utterance by jointly encoding utterance and speaker state. Each state also serves as a speaker-specific utterance representation. Attending on these states facilitates the inter-speaker and inter-utterance dependencies to produce improved context representation. The current utterance uₜ changes the speaker's state from qₛ₍ᵤ_{ₜ}_{),t-1} to qₛ₍ᵤ_{ₜ}_{),t}. This change may be captured with GRU cell [symbol] with output size [symbol] , using uₜ and qₛ₍ᵤ_{ₜ}_{),t-1}:gₜ= [symbol] (gₜ₋₁, (uₜ⊕qₛ₍ᵤ_{ₜ}_{),t-1})), where [symbol] is the size of the global state vector, [symbol] is the size of speaker state vector [symbol] ∈ [symbol] , [symbol] ∈ [symbol] , [symbol] ∈ [symbol] , qₛ₍ᵤ_{ₜ}_{),t-1}∈ [symbol] , tₜ, tₜ₋₁∈ [symbol] , [symbol] is speaker state size, and ⊕ represents concatenation.

Speaker State (Speaker GRU) 606, such as speaker-state modeling keeps track of the state of individual speakers using fixed size vectors q₁, q₂, . . . , q_{M} throughout the conversation. These states are representative of the speakers' state in the conversation, relevant to cognitive state/emotion classification. These states may be updated based on the current (at time t) role of a participant in the conversation, which is either speaker or listener, and the incoming utterance uₜ. These state vectors are initialized with null vectors for all the participants. The main purpose of this module is to ensure that the model is aware of the speaker of each utterance and handle it accordingly.

GRU cells [symbol] 608 may be used to update the states and representations. Each GRU cell computes a hidden state defined as hₜ=GRU*(hₜ₋₁, xₜ), where xₜ is the current input and hₜ₋₁ is the previous GRU state. hₜ also serves as the current GRU output. GRUs are efficient networks with trainable parameters: W*_{,{h,x}}^{{r,z,c}} and b*^{{r,z,c}}. *, {h,x}

Update of the speaker-state 606 may be performed by Speaker GRU 608. A speaker usually frames their response based on the context, which is the preceding utterances in the conversation. Hence, the context cₜ relevant to the utterance uₜ may be captured as follows:

$$α = softmax ( {u}_{t}^{T} {W}_{α} [ {g}_{1} , {g}_{2} , … , {g}_{t - 1} ] ) ,$$

$$softmax ( x ) = [ {e}^{{x}_{1}} / {\sum }_{i} {e}^{{x}_{i}} , {e}^{{x}_{2}} / {\sum }_{i} {e}^{{x}_{i}} , … ] ,$$

$${c}_{t} = {α [ {g}_{1} , {g}_{2} , … , {g}_{t - 1} ]}^{T} ,$$

where g₁, g₂, . . . , gₜ₋₁ are the preceding t−1 global states (gᵢ∈ [symbol] ), W_{α}∈ [symbol] , α^{T}∈ [symbol] ^{(t-1)}, and cₜ∈ [symbol] . In the first equation above, attention scores a are calculated over the previous global states representative of the previous utterances. This assigns higher attention scores to the utterances relevant to uₜ. Finally, in the third equation above, the context vector cₜ is calculated by pooling the previous global states with α.

GRU cell [symbol] 608 may be used update the current speaker state qₛ₍ᵤ_{ₜ}_{),t-1} to the new state qₛ₍ᵤ_{ₜ}_{),t} based on incoming utterance uₜ and context cₜ using GRU cell [symbol] 608 of output size [symbol] : qₛ₍ᵤ_{ₜ}_{),t}= [symbol] (qₛ₍ᵤ_{ₜ}_{),t-1}, (uₜ⊕cₜ)), where [symbol] ∈ [symbol] , [symbol] ∈ [symbol] , [symbol] ∈ [symbol] , qₛ₍ᵤ_{ₜ}_{),t}, qₛ₍ᵤ_{ₜ}_{),t-1}∈ [symbol] , This encodes the information on the current utterance along with its context from the global GRU 604 into the speaker's state qₛ₍ᵤ_{ₜ}₎ which helps in cognitive state/emotion classification down the line.

The Listener state models the listeners' change of state due to the speaker's utterance. Embodiments may use listener state update mechanisms such as: Simply keep the state of the listener unchanged, that is ∀i≠+s(uₜ), q_{i,t}=q_{i,t-1}. Embodiments may use listener state update mechanisms such as: Employ another GRU cell [symbol] to update the listener state based on listener visual cues (facial expression) v_{i,t} and its context cₜ, as ∀i≠s(uₜ) [symbol] (q_{i,t-1}, (v_{i,t}⊕cₜ), where v_{i,t}∈ [symbol] ^{D}^{ᵥ}, [symbol] ∈ [symbol] , [symbol] ∈ [symbol] , and [symbol] ∈ [symbol] . Listener visual features of speaker i at time t v_{i,t} are extracted using a model introduced by Arriaga, Valdenegro-Toro, and Ploger (2017), pretrained on FER2013 dataset, where feature size D_{V}=7.

Cognitive State/Emotion Representation (Emotion GRU) 610 may infer the relevant representation eₜ of utterance uₜ from the speaker's state qₛ₍ᵤ_{ₜ}_{),t} and the cognitive state/emotion representation of the previous utterance eₜ₋₁. Since context is important to the cognitive state/emotion of the incoming utterance qₛ₍ᵤ_{ₜ}_{),t} feeds fine-tuned relevant contextual information from other the speaker states qₛ₍ᵤ_{_{<t}}_{),<t} into the cognitive state/emotion representation eₜ. This establishes a connection between the speaker state and the other speaker states. Hence, eₜ may be modeled with a GRU cell (GRU_{ε}) with output size D_{ε} as eₜ=GRU_{ε}(eₜ₋₁, qₛ₍ᵤ_{ₜ}_{),t}), where D_{ε} is the size of cognitive state/emotion representation vector, e_{{t,t-1}}∈ [symbol] ^{D}^{_{ε}}, W_{ε,H}^{{R,Z,C}}∈ [symbol] ^{D}^{_{ε}}^{×D}^{_{ε}}, W_{ε,x}^{{r,z,c}}∈ [symbol] , and b_{ε}^{{r,z,c}}∈ [symbol] ^{D}^{_{ε}}.

Embodiments may perform Cognitive State/Emotion Classification using, for example, a two-layer perceptron with a final softmax layer to calculate c=6 emotion-class probabilities from cognitive state/emotion representation eₜ of utterance uₜ and then we pick the most likely cognitive state/emotion class:

$${l}_{t} = R e L U ( {W}_{l} {e}_{t} + {b}_{l} ) ,$$

$${𝒫}_{t} = softmax ( {W}_{smax} {l}_{t} + {b}_{smax} ) ,$$

$${\hat{y}}_{t} = {arg max}_{i} ( {𝒫}_{t} [ i ] ) ,$$

where Wₗ∈ [symbol] ^{D}^{ₗ}^{×D}^{_{ε, b}}ₗ∈ [symbol] ^{D}^{ₗ}, Wₛₘₐₓ∈ [symbol] ^{c×D}^{ₗ}, bₛₘₐₓ∈ [symbol] ^{c}, [symbol] ₜ∈ [symbol] ^{c}, and ŷₜ is the predicted label for utterance uₜ.

Embodiments may be trained using categorical cross-entropy along with L2-regularization as the measure of loss (L) during training:

$$L = - \frac{1}{{\sum }_{s = 1}^{N} c ( s )} {\sum }_{i = 1}^{N} {\sum }_{j = 1}^{c ( i )} log {𝒫}_{i , j} [ {y}_{i , j} ] + λ {‖ θ ‖}_{2} ,$$

where N is the number of samples/dialogues, c(i) is the number of utterances in sample i, [symbol] _{i,j} is the probability distribution of cognitive state/emotion labels for utterance j of dialogue i, y_{i,j} is the expected class label of utterance j of dialogue i, λ is the L2-regularizer weight, and θ is the set of trainable parameters
where

$$θ = \{ {W}_{α} , {W}_{𝒫 , \{ h , x \}}^{\{ r , z , c \}} , {b}_{𝒫}^{\{ r , z , c \}} , {W}_{𝒢 , \{ h , x \}}^{\{ r , z , c \}} , {b}_{𝒢}^{\{ r , z , c \}} , {W}_{ℰ , \{ h , x \}}^{\{ r , z , c \}} , {b}_{ℰ}^{\{ r , z , c \}} , {W}_{l} , {b}_{l} , {W}_{smax} , {b}_{sm ax} \} .$$

Embodiments may use stochastic gradient descent based Adam (Kingma and Ba 2014) optimizer to train our network. Hyperparameters are optimized using grid search.

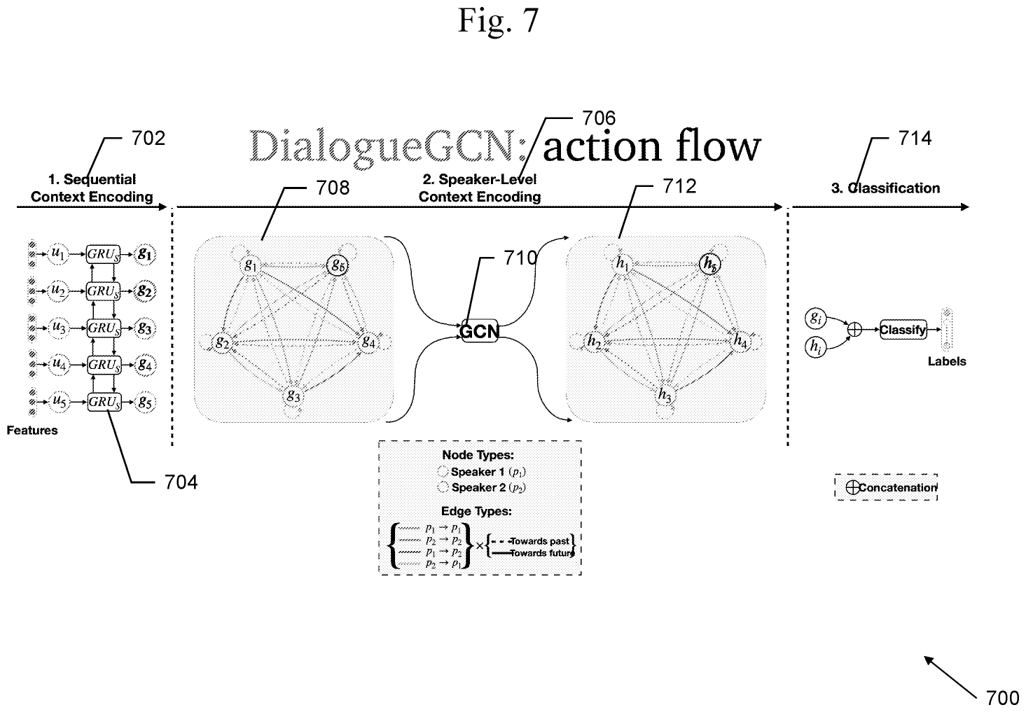

An example of how dialogue is represented as a graph, followed by a graph convolutional layer to get convoluted features which are used to obtain depression score is shown in FIG. 7. This example uses a graph convolutional network, such as implemented by DialogueGCN process 700 to track the conversation as the utterances are being fed, representing a global state, a speaker state indicating a profile of each individual speaker, and a disorder state. Utterances may be fed to process 700 and, at 702, Sequential Context Encoding may be performed.

Since conversations are sequential by nature, contextual information flows along that sequence. The conversation may be fed to a bidirectional gated recurrent unit (GRU) to capture this contextual information: for i=1, 2, . . . , N, where uᵢ and gᵢ are context-independent and gᵢ= [symbol] (g_{i(+,-)1}, uᵢ) sequential context-aware utterance representations, respectively.

Since the utterances are encoded irrespective of their speakers, this initial encoding scheme is speaker agnostic, as opposed to the state of the art, DialogueRNN (Majumder et al., 2019). At 706, speaker-level context encoding may be performed.

At 708, a directed graph may be created from the sequentially encoded utterances to capture this interaction between the participants. A local neighborhood based convolutional feature transformation process, such as graph convolutional network (GCN) 710 may be used to create the enriched speaker-level contextually encoded features 712. The framework is detailed here.

First, the following notation is introduced: a conversation having N utterances is represented as a directed graph [symbol] =(V,ε, [symbol] ,W), with vertices/nodes vᵢ∈V, labeled edges (relations) rᵢⱼ∈ε where r∈ [symbol] is the relation type of the edge between vᵢ and vⱼ and αᵢⱼ is the weight of the labeled edge rᵢⱼ, with 0<αᵢⱼ≤1, where αᵢⱼ∈W and i,j∈[1, 2, . . . , N].

At 708, the graph may be constructed from the utterances as follows: Vertices: Each utterance in the conversation may represented as a vertex vᵢ∈V in [symbol] . Each vertex vᵢ is initialized with the corresponding sequentially encoded feature vector gᵢ, for all i∈[1, 2, . . . , N]. This vector may be denoted the vertex feature. Vertex features are subject to change downstream, when the neighborhood based transformation process is applied to encode speaker-level context.

Edges: Construction of the edges ε depends on the context to be modeled. For instance, if each utterance (vertex) is contextually dependent on all the other utterances in a conversation (when encoding speaker level information), then a fully connected graph would be constructed. That is, each vertex is connected to all the other vertices (including itself) with an edge. However, this results in O(N²) number of edges, which is computationally very expensive for graphs with large numbers of vertices. A more practical solution is to construct the edges by keeping a past context window size of p and a future context window size of f. In this scenario, each utterance vertex vᵢ has an edge with the immediate p utterances of the past: vᵢ₋₁, vᵢ₋₂, . . . vᵢ₋ₚ, f utterances of the future: vᵢ₊₁, vᵢ₊₂, . . . v_{i+f} and itself: vᵢ. For example, a past context window size of 10 and future context window size of 10 may be used. As the graph is directed, two vertices may have edges in both directions with different relations.

The edge weights may be set using a similarity based attention module. The attention function is computed in a way such that, for each vertex, the incoming set of edges has a sum total weight of 1. Considering a past context window size of p and a future context window size of f, the weights are calculated as follows, αᵢⱼ=softmax(gᵢ^{T}Wₑ[gᵢ₋ₚ, . . . , g_{i+f}), for j=I−p, . . . , i=f. This ensures that, vertex vᵢ which has incoming edges with vertices vᵢ₋ₚ, . . . , v_{i+f} (as speaker level context) receives a total weight contribution of 1.

In embodiments, the Speaker-Level Context Encoding 706 may have the form of a graphical network to capture speaker dependent contextual information in a conversation. Effectively modelling speaker level context requires capturing the inter-dependency.

Relations: The relation r of an edge rᵢⱼ is set depending upon two aspects: speaker dependency and temporal dependency.

Speaker dependency relation depends on both the speakers of the constituting vertices: pₛ₍ᵤ_{ᵢ}₎ (speaker of vᵢ) and pₛ₍ᵤ_{ⱼ}₎ (speaker of vⱼ). Temporal dependency also depends upon the relative position of occurrence of uᵢ and uⱼ in the conversation: whether uᵢ is uttered before uⱼ or after. If there are M distinct speakers in a conversation, there can be a maximum of M (speaker of uᵢ)*M (speaker of uⱼ)*2 (uᵢ occurs before uⱼ or after)=2M² distinct relation types r in the graph [symbol] .

Each speaker in a conversation is uniquely affected by each other speaker, hence explicit declaration of such relational edges in the graph helps in capturing the inter-dependency and self-dependency among the speakers, which in succession would facilitate speaker-level context encoding.

As an illustration, let two speakers p₁, p₂ participate in a dyadic conversation having 5 utterances, where u₁, u₃, u₅ are uttered by p₁ and u₂, u₄ are uttered by p₂. Considering a fully connected graph, the edges and relations will be constructed as shown in Table 1.

TABLE 1

| Relation | pₛ(uᵢ), pₛ(uⱼ) | i < j | (i, j) |
|---|---|---|---|
| 1 | p₁, p₁ | Yes | (1, 3), (1, 5), (3, 5) |
| 2 | p₁, p₁ | No | (1, 1), (3, 1), (3, 3) (5, 1), |
| | | | (5, 3), (5, 5) |
| 3 | p₂, p₂ | Yes | (2, 4) |
| 4 | p₂, p₂ | No | (2, 2), (4, 2), (4, 4) |
| 5 | p₁, p₂ | Yes | (1, 2), (1, 4), (3, 4) |
| 6 | p₁, p₂ | No | (3, 2), (5, 2), (5, 4) |
| 7 | p₂, p₁ | Yes | (2, 3), (2, 5), (4, 5) |
| 8 | p₂, p₁ | No | (2, 1), (4, 1), (4, 3) |

In Table 1, pₛ(uᵢ) and pₛ(uⱼ) denote the speaker of utterances uᵢ and uⱼ, respectively. Two distinct speakers in the conversation implies 2*M²=2*2²=8 distinct relation types. The rightmost column denotes the indices of the vertices of the constituting edge that was the relation type indicated by the leftmost column.

GCN 710 may perform feature transformation to transform the sequentially encoded features using the graph network. The vertex feature vectors (gᵢ) are initially speaker independent and thereafter transformed into a speaker dependent feature vector using a two-step graph convolution process. Both of these transformations may be understood as special cases of a basic differentiable message passing method. In the first step, a new feature vector hᵢ⁽¹⁾ is computed for vertex vᵢ by aggregating local neighborhood information (in this case neighbor utterances specified by the past and future context window size) using the relation specific transformation:

$${h}_{i}^{( 1 )} = σ ( {\sum }_{r ∈ ℛ} {\sum }_{j ∈ {N}_{i}^{r}} \frac{{α}_{i j}}{{c}_{i , r}} {W}_{r}^{( 1 )} {g}_{j} + {α}_{i i} {W}_{0}^{( 1 )} {g}_{i} ) , for i = 1 , 2 , … , N ,$$

where, αᵢⱼ and αᵢᵢ are the edge weights, Nᵢ^{r} denotes the neighboring indices of vertex i under relation r∈ [symbol] . Then c_{i,r} is a problem specific normalization constant which either can be set in advance, such that, c_{i,r}=|Nᵢ^{r}|, or can be automatically learned in a gradient based learning setup. Also, σ is an activation function such as ReLU, Wᵣ⁽¹⁾ and W₀⁽¹⁾ are learnable parameters of the transformation.

In the second step, another local neighborhood based transformation is applied over the output of the first step,

$${h}_{i}^{( 2 )} = σ {\sum }_{j ∈ {N}_{i}^{r}} {W}^{( 2 )} {h}_{j}^{( 1 )} + {W}_{0}^{( 2 )} {h}_{i}^{( 1 )} , for i = 1 , 2 , … , N ,$$

where W⁽²⁾ and W₀⁽²⁾ are parameters of these transformation and σ is the activation function. This stack of transformations effectively accumulates the normalized sum of the local neighborhood (features of the neighbors) i.e. the neighborhood speaker information for each utterance in the graph. The self-connection ensures self-dependent feature transformation.

Cognitive State/Emotion classifier 714 may then be applied to the contextually encoded feature vectors gⱼ (from sequential encoder 702) and hᵢ⁽²⁾ (from speaker-level encoder 706), which are concatenated and a similarity-based attention mechanism is applied to obtain the final utterance representation:

$${h}_{i} = [ {g}_{i} , {h}_{i}^{( 2 )} ] ,$$

$${β}_{i} = softmax ( {h}_{i}^{T} {W}_{β} [ {h}_{1} , {h}_{2} , … , {h}_{N} ] ) ,$$

$${\tilde{h}}_{i} = {{β}_{i} [ {h}_{1} , {h}_{2} , … , {h}_{N} ]}^{T} .$$

Finally, the utterance is classified using a fully-connected network:

$${l}_{i} = ReLU ( {W}_{l} {\tilde{h}}_{i} + {b}_{l} ,$$

$${𝒫}_{i} = softmax ( {W}_{sm ax} {l}_{i} + {b}_{sm ax} ) ,$$

$${\hat{y}}_{i} = {arg max}_{k} ( {𝒫}_{i} [ k ] ) .$$

The artificial intelligence and/or machine learning models involved in, for example, DialogueGCN may be trained using, for example categorical cross-entropy along with L2-regularization as the measure of loss (L) during training:

$$L = - \frac{1}{{\sum }_{s = 1}^{N} c ( s )} {\sum }_{i = 1}^{N} {\sum }_{j = 1}^{c ( i )} log {𝒫}_{i , j} [ {y}_{i , j} ] + λ {‖ θ ‖}_{2} ,$$

where N is the number of samples/dialogues, c(i) is the number of utterances in sample i, [symbol] _{i,j} is the probability distribution of cognitive state/emotion labels for utterance j of dialogue i, y_{i,j} is the expected class label of utterance j of dialogue i, A is the L2-regularizer weight, and θ is the set of all trainable parameters. A stochastic gradient descent based Adam optimizer may be used to train the network. Hyperparameters may be optimized using grid search.

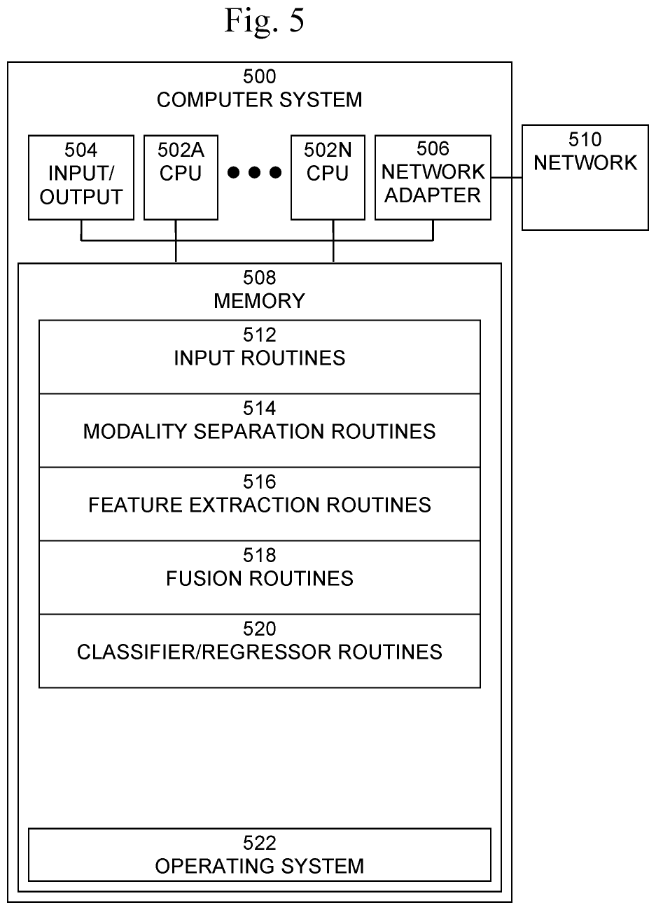

An exemplary block diagram of a computer system 500, in which processes and components involved in the embodiments described herein may be implemented, is shown in FIG. 5. Computer system 500 may be implemented using one or more programmed general-purpose computer systems, such as embedded processors, systems on a chip, personal computers, workstations, server systems, and minicomputers or mainframe computers, or in distributed, networked computing environments. Computer system 500 may include one or more processors (CPUs) 502A-502N, input/output circuitry 504, network adapter 506, and memory 508. CPUs 502A-502N execute program instructions in order to carry out the functions of the present communications systems and methods. Typically, CPUs 502A-502N are one or more microprocessors, such as an INTEL CORE® processor. FIG. 5 illustrates an embodiment in which computer system 500 is implemented as a single multi-processor computer system, in which multiple processors 502A-502N share system resources, such as memory 508, input/output circuitry 504, and network adapter 506. However, the present communications systems and methods also include embodiments in which computer system 500 is implemented as a plurality of networked computer systems, which may be single-processor computer systems, multi-processor computer systems, or a mix thereof.

Input/output circuitry 504 provides the capability to input data to, or output data from, computer system 500. For example, input/output circuitry may include input devices, such as keyboards, mice, touchpads, trackballs, scanners, analog to digital converters, etc., output devices, such as video adapters, monitors, printers, etc., and input/output devices, such as, modems, etc. Network adapter 506 interfaces device 500 with a network 510. Network 510 may be any public or proprietary LAN or WAN, including, but not limited to the Internet.

Memory 508 stores program instructions that are executed by, and data that are used and processed by, CPU 502 to perform the functions of computer system 500. Memory 508 may include, for example, electronic memory devices, such as random-access memory (RAM), read-only memory (ROM), programmable read-only memory (PROM), electrically erasable programmable read-only memory (EEPROM), flash memory, etc., and electro-mechanical memory, such as magnetic disk drives, tape drives, optical disk drives, etc., which may use an integrated drive electronics (IDE) interface, or a variation or enhancement thereof, such as enhanced IDE (EIDE) or ultra-direct memory access (UDMA), or a small computer system interface (SCSI) based interface, or a variation or enhancement thereof, such as fast-SCSI, wide-SCSI, fast and wide-SCSI, etc., or Serial Advanced Technology Attachment (SATA), or a variation or enhancement thereof, or a fiber channel-arbitrated loop (FC-AL) interface.

The contents of memory 508 may vary depending upon the function that computer system 500 is programmed to perform. In the example shown in FIG. 5, exemplary memory contents are shown representing routines and data for embodiments of the processes described above. However, one of skill in the art would recognize that these routines, along with the memory contents related to those routines, may not be included on one system or device, but rather may be distributed among a plurality of systems or devices, based on well-known engineering considerations. The present systems and methods may include any and all such arrangements.

In the example shown in FIG. 5, memory 508 may include input routines 512, modality separation routines 514, feature extraction routines 516, fusion routines 518, classifier/regressor routines 520, and operating system 522. Input routines 512 may include software to obtain an input stream, as described above. Modality separation routines 514 may include software to separate features from each channel/modality, as described above. Feature extraction routines 516 may include software to extract features from each channel/modality, as described above. Fusion routines 518 may include software to perform multimodal fusion of the extracted features, as described above. Classifier/regressor routines 520 may include software to perform speaker-specific detection of a mental disorder, as described above. Operating system 522 may provide overall system functionality.

As shown in FIG. 5, the present communications systems and methods may include implementation on a system or systems that provide multi-processor, multi-tasking, multi-process, and/or multi-thread computing, as well as implementation on systems that provide only single processor, single thread computing. Multi-processor computing involves performing computing using more than one processor. Multi-tasking computing involves performing computing using more than one operating system task. A task is an operating system concept that refers to the combination of a program being executed and bookkeeping information used by the operating system. Whenever a program is executed, the operating system creates a new task for it. The task is like an envelope for the program in that it identifies the program with a task number and attaches other bookkeeping information to it. Many operating systems, including Linux, UNIX®, OS/2®, and Windows®, are capable of running many tasks at the same time and are called multitasking operating systems. Multi-tasking is the ability of an operating system to execute more than one executable at the same time. Each executable is running in its own address space, meaning that the executables have no way to share any of their memory. This has advantages, because it is impossible for any program to damage the execution of any of the other programs running on the system. However, the programs have no way to exchange any information except through the operating system (or by reading files stored on the file system). Multi-process computing is similar to multi-tasking computing, as the terms task and process are often used interchangeably, although some operating systems make a distinction between the two.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

1. A method, implemented in a computer system comprising a processor, memory accessible by the processor, and computer program instructions stored in the memory and executable by the processor, the method comprising:
receiving input data relating to communications among persons, the input data comprising a plurality of modalities;
extracting features relating to the plurality of modalities from the received input data;
performing multimodal fusion on the extracted features, wherein the multimodal fusion is performed on at least some of the features relating to individual modalities and on at least some combinations of features relating to a plurality of modalities;
classifying the fused features using a trained model for detection of at least one mental disorder; and
generating a representation of a disorder state based on the classified fused features.

2. The method of claim 1, wherein the plurality of modalities comprises text information, audio information, and video information.

3. The method of claim 2, wherein the multimodal fusion is performed on at least some of the text information, audio information, video information, text-audio information, text-video information, audio-video information, and text-audio-video information.

4. The method of claim 3, wherein the mental disorder is one of depression, anxiety, suicidal ideation, and post-traumatic stress disorder.

5. The method of claim 3, wherein the mental disorder is depression and the representation of the disorder state is one of a predicted PHQ-9 and a CES-D Depression Score.

6. The method of claim 3, wherein the persons are any of at least one of age, gender, race, nationality, ethnicity, culture, and language.

7. The method of claim 3, wherein the method is implemented as a stand-alone application, is integrated with a telemedicine/telehealth platform, is integrated with other software, or is integrated with other applications/marketplaces that provide access to counselors and therapy.

8. The method of claim 3, wherein the method is used for at least one of screening in clinical settings (ER visits, primary care, pre and post-surgery), validating clinical observations (provision of 2nd opinions, expediting complicated diagnostic paths, verifying clinical determinations), screening in the field (at home, school, workplace, in the field), virtual follow up via telehealth scenarios (synchronous-video call with patient, asynchronous-video messages), self-screening for consumer use (triage channels, self-administered assessments, referral mechanisms), screening through helplines (suicide prevention, employee assistance).

9. A system comprising a processor, memory accessible by the processor, and computer program instructions stored in the memory and executable by the processor to perform:
receiving input data relating to communications among persons, the input data comprising a plurality of modalities;
extracting features relating to the plurality of modalities from the received input data;
performing multimodal fusion on the extracted features, wherein the multimodal fusion is performed on at least some of the features relating to individual modalities and on at least some combinations of features relating to a plurality of modalities;
classifying the fused features using a trained model for detection of at least one mental disorder; and
generating a representation of a disorder state based on the classified fused features.

10. The system of claim 9, wherein the plurality of modalities comprises text information, audio information, and video information.

11. The system of claim 10, wherein the multimodal fusion is performed on at least some of the text information, audio information, video information, text-audio information, text-video information, audio-video information, and text-audio-video information.

12. The system of claim 11, wherein the mental disorder is one of depression, anxiety, suicidal ideation, and post-traumatic stress disorder.

13. The system of claim 11, wherein the mental disorder is depression and the representation of the disorder state is one of a predicted PHQ-9 and a CES-D Depression Score.

14. The system of claim 11, wherein the persons may be of any of at least one of age, gender, race, nationality, ethnicity, culture, and language.

15. The system of claim 11, wherein the method is implemented as a stand-alone application, is integrated with a telemedicine/telehealth platform, is integrated with other software, or is integrated with other applications/marketplaces that provide access to counselors and therapy.

16. The system of claim 11, wherein the method is used for at least one of screening in clinical settings (ER visits, primary care, pre and post-surgery), validating clinical observations (provision of 2nd opinions, expediting complicated diagnostic paths, verifying clinical determinations), screening in the field (at home, school, workplace, in the field), virtual follow up via telehealth scenarios (synchronous—video call with patient, asynchronous—video messages), self-screening for consumer use (triage channels, self-administered assessments, referral mechanisms), screening through helplines (suicide prevention, employee assistance).

17. A computer program product comprising a non-transitory computer readable storage having program instructions embodied therewith, the program instructions executable by a computer, to cause the computer to perform a method comprising:
receiving input data relating to communications among persons, the input data comprising a plurality of modalities;
extracting features relating to the plurality of modalities from the received input data;
performing multimodal fusion on the extracted features, wherein the multimodal fusion is performed on at least some of the features relating to individual modalities and on at least some combinations of features relating to a plurality of modalities;
classifying the fused features using a trained model for detection of at least one mental disorder; and
generating a representation of a disorder state based on the classified fused features.

18. The computer program product of claim 17, wherein the plurality of modalities comprises text information, audio information, and video information.

19. The computer program product of claim 18, wherein the multimodal fusion is performed on at least some of the text information, audio information, video information, text-audio information, text-video information, audio-video information, and text-audio-video information.

20. The computer program product of claim 19, wherein the mental disorder is one of depression, anxiety, suicidal ideation, and post-traumatic stress disorder.

21.-24. (canceled)
